(19) United States
(12) Patent Application Publication
(10) Pub. No.: US 2015/0297190 A1
(43) Pub. Date: Oct. 22, 2015

(54) DEVICE AND METHOD OF MEDICAL IMAGING

(71) Applicant: Ted Selker, Palo Alto, CA (US)

(72) Inventor: Ted Selker, Palo Alto, CA (US)

(21) Appl. No.: 14/255,810

(22) Filed: Apr. 17, 2014

(51) Int. Cl. A61B 8/08; A61B 5/05; A61B 5/00

(52) U.S. Cl. CPC A61B 8/5292; A61B 5/0035; A61B 5/05

(57) ABSTRACT

Device and method of imaging a limb, such as an arm or leg, by scanning using both an optical camera and an ultrasound sensor, where the devices are electronically or mechanically linked such they both scan a portion of the limb, and the images displayed from the two sensors are visually aligned. Scanning may be in one or both of the longitudinal plane and the axis of the limb, such that most of the surface of the limb may be imaged. Stitched images from the sensors are visually aligned relative to the anatomical features of the limb. The limb is placed in a receptacle, typically with an open top, containing a transparent fluid through which both sensors operate. Either the sensors move (scan) relative to the fixed receptacle or the receptacle rotates, such as on a turntable.

The field of this invention is medical imaging. More specifically, the field is the combined use of ultrasound and visual imaging.

### BACKGROUND OF THE INVENTION

Medical imaging technologies are primary visual, x-ray, ultra-sound, X-ray computed tomography (“CAT scan”) and MRI. Each of these has specific advantages and disadvantages. Typically, these different technologies are not computer combined, although a physician may use more than one as part of diagnoses or to assist in treatment.

Thus, physicians do not currently have the ability to overlay or accurately anatomically align the images from multiple technologies in order to combine the strengths of different technologies while overcoming the weaknesses.

A weakness of ultra-sound is that its normal use is to manually place and move a single-point transducer around a small area of skin, creating a series of narrow video images, each image comprising a cone-shaped view into the body from the transducer. Such images each provide a highly limited view of internal anatomy.

A weakness of x-rays is the exposure of the patient to undesirable radiation. This weakness is especially acute for children and pregnant women. The desire to not use x-rays for these patients makes accurate diagnosis of bone breaks, particularly non-displacement breaks, problematic.

A weakness of MRI and CAT scans is their high-cost, time-consuming diagnosis, and emotional trauma to the subject, particularly children.

### SUMMARY OF THE INVENTION

Embodiments of this invention overcome many of the above weaknesses.

In one embodiment, a limb, such as a leg or arm, is placed in a bucket-like receptacle. Two sensors, one a camera and the second an ultrasound transducer, are scanned around the limb, ideally in both the transverse plane and parallel to the axis of the limb. That is, the sensors are moved to cover a fraction (or most, or all) of the interior surface of the bucket, imaging towards the proximal portion of the limb, such that a suitable fraction of the limb is imaged, “from all sides.”

In addition, the sensors are linked, either mechanically, electronically, or computationally, such that their separate imageries of the limb are linked relative to the same anatomical limb location.

In some embodiments, the images from the two sources are computationally “stitched,” such that one or more continuous images are available for viewing, each such continuous image built out of a plurality of individual images captured by the sensors.

In some embodiments, the images from the two sources are displayed together, such as overlaid, merged, side-by-side, or alternately so that the physician may readily see how the now visible features generated by one sensor compare with the now visible features generated by the second sensor.

Some embodiments move the sensors within the bucket-like receptacle, while the receptacle is relatively fixed; whereas other embodiments rotate the receptacle, move the receptacle axially, or combination of these motion-related embodiments.

Some embodiments use a magnetic linkage between a driving source, through the receptacle, to one or more sensors. Driving mechanisms may be belt or chain driven, screw driven, stepping motor (including micro-step) driven, servomotor driven, or pneumatically driven.

Some embodiments use a fluid or gel within the receptacle between the limb and the sensors.

Some embodiments enclose the limb in a protective sheath prior to imaging.

Some embodiments move one or more sensors radially to the limb to maintain a desired distance between the limb surface and the sensor.

Some embodiments change the focal length (“zoom”) of the camera to maintain a desired field of view of the proximal surface of the limb.

One application of this technology is an alternative to x-ray, where it is not desirable to expose the patient to radiation.

One application of this technology is to diagnose bone breaks, particularly in children.

Another application of this technology is to clearly associate below-the skin pathologies with visible skin pathologies.

Another application of this technology is to more accurately assess trauma damage to limbs, particularly for children.

Another application of this technology is an alternative to MRI and CAT scans for fieldwork where MRI or CAT scans may not be available, due to cost, distance, training, or other reasons.

Having a child place a foot, leg, hand or arm in gel-filled bucket, in a doctor's office, with a parent or caregiver close by, is a rapid and non-traumatic way for the child, physician and caregiver to receive an immediate diagnosis.

### BRIEF DESCRIPTION OF THE DRAWINGS

FIG. 1 shows a schematic of one embodiment.

FIGS. 2A, 2B, 2C and 2D shows alternative embodiments of comparing images from two different sensors.

FIG. 3 shows another schematic view of a leg in a receptacle, with a turntable.

FIG. 4 shows two preferred axes of scanning.

### DETAILED DESCRIPTION

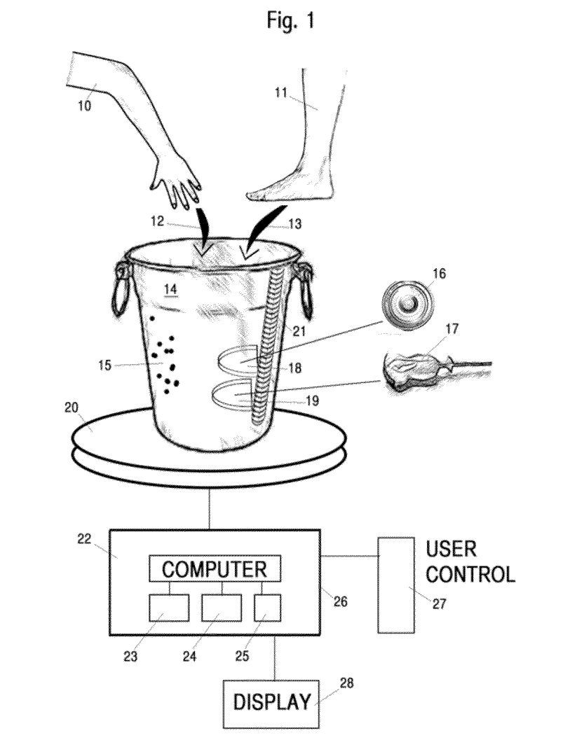

FIG. 1 shows key elements in one embodiment for both a device and a method for this invention. This Figure is purely schematic in nature.

A limb, such as an arm, hand, or both, 10, may be placed, 12, in a bucket-like receptacle, 14. Another example of a limb is a leg, foot, or both, 11, also shown placed, 13, into the receptacle, 14. The limb, 10 or 11, may be enclosed in thin, protective sheath, such a thin latex or latex-free rubber “condom,” so as to both protect a damaged limb and provide some level of sanitation against contamination. Such a sheath is not shown in this Figure. A disposable insert may be used in the receptacle, which may then hold contamination from the limb, and then the insert may be discarded for sanitation. Such a disposable insert is not shown in this Figure.

The receptacle comprises two sensors, a camera, 16, and an ultrasound transducer, 17. These are shown schematically in this Figure. In one embodiment, actual positions of the camera, 16 and the ultrasound transducer, 17, are shown as 18 and 19, respectively. Typically, the two sensors are mounted proximal to the interior wall of the receptacle, 14, and face the center of the receptacle so as to face the surface of the limb, 10 or 11, placed into the receptacle for imaging.

Ideally, there are two axis of motion used for imaging, although in some embodiments only one axis may be used. In some embodiments, a third, axis, distance from the surface of the limb, may also be used. We refer to the first axis as rotation in the transverse plane of the limb. Motion along this axis may be accomplished by rotating the sensors around the interior of the receptacle, 14; or it may be accomplished by rotating the receptacle, as shown here by the use of a turntable, 20, on which the receptacle rests. In another embodiment, the receptacle, 14, remains rotationally fixed relative to the limb, and the turntable, 20, rotates and is coupled through the receptacle to the sensors 16 and 17, magnetically, using either permanent magnets, electro-magnets, or both. Magnetic coupling is not shown in this Figure. The purpose of motion in this axis is to image areas along a transverse plane of the limb.

The second axis of motion, parallel to the axis of the limb, may be viewed as “up and down” along the inside of the receptacle, 14, as shown with the orientation of this Figure. The purpose of this axis is to image some potion of the limb between a more distal area and a more medial area of the limb. One embodiment uses a screw drive, 21, for this axis.

Motions for the turntable, screw drive, or other methods of movement and coupling include the use of stepper motors, including micro-step, or servomotors, or pneumatic drive. Couplings may include belts; chains; pulleys; gears; metal, plastic or rubber wire-drive; or direct coupling, or other methods. Stepper motors have the advantage that the location of the sensors is known by counting steps. Servos may be lower cost, and position location sensors, such as one or more accelerometers, visible markings inside the receptacle, a coded position wheel with a magnetic or optical “tic” sensor, are practical embodiments. Motor drives and position sensors are not shown in this Figure. The sensors or sensor assembly may be placed on a track for one or both axis, and pushed or pulled along the track. A belt is good way to pull a sensor assembly—not shown in this Figure. A rubber or similar material capstan, in contact with a portion of the receptacle, is one method of spinning the receptacle. A belt surrounding the periphery or an attached wheel is one method of spinning the receptacle.

Mechanical mechanisms for moving one or more sensors, or the receptacle, are well known in the art and thus will be described minimally herein so as to establish complete enablement and disclosure herein for functional claiming, without excessive and unnecessary verbiage and with no loss of completeness.

The receptacle, 14, may be filled with a gel, 15, or other fluid to improve the performance of the ultrasound transducer. The transducer may be spring loaded or driven so that it against the skin of the limb, 10 or 11, in some embodiments. In other embodiments, it is not placed against the skin, but provided ultrasound imaging through the gel, 15. In one embodiment the gel, 15, is transparent so that the camera, 16, may view the skin of the limb, 10 or 11, through the gel, 15.

Some embodiments include illumination through the receptacle, 14, or inside the receptacle for use by the camera. The illumination may be visible light, IR light, UV light, or combinations. Such illumination sources are not shown in the Figure.

Some embodiments use a “double bucket” as an alternative to a liner or insert. The limb and gel or liquid is placed inside the inner bucket with the sensors and at least some mechanisms located between the two buckets. The inner bucket is transparent to both the ultrasound and visible light. The ultrasound sensor may operate against the inner bucket. The inner bucket may or may not be disposable. It may or may not be readily removable.

Some embodiments use infrared (IR) light, particularly deep IR light in order to see beneath the surface of the skin. Such IR sensors, cameras, light sources and imaging are included in all discussions herein of “visible” light.

Some embodiments use x-ray or MRI sensors in place of or in addition to ultrasound sensors. CT, PET, X-ray, DSA, and isotope, or also alternative or additional sensors.

Some embodiments use more than two linked scanners, and present more than two images for comparison on the display.

Some embodiments permit manual scanning in one or both axis. Some embodiments permit the starting position, ending position, or both (in either or both axes), to be manually set.

The ultrasound sensor may be elastomeric, or may include a piezoelectric transducer. The shape of the transducer head, or an acoustical matching layer for the head, may conform to the inner bucket, or to the free gel or fluid.

In one embodiment images are presented in 3D. Note that this is particularly simple since the set of longitudinal plane images inherently has a large number of stereo image pairs. For this embodiment the display may be a 3D display. Output may be one or more of many 3D file formats, such as 3dmlw; edxml; AR (Ashlar-Vellum Argon); CO (Alshar-Vellum Cobalt); SCAD; SCDOC; SLDASM; SLDDRAW; SLDPRT; and XE (Ashlar-Vellum Xenon).

A computer, 26 is used to record the electronic images from the camera, 16 and the ultrasonic transducer, 17, as well as to drive the axis of movement, such as via the turntable, 20, and the screw, 21. The computer typically includes a programmable processor, 22, stored program(s) for accomplishing the embodiments of this invention, 23, memory for temporarily (but not transitory) storing the digital images from the sensors, 24, and input/output circuitry, 25, as those in the art well-understand.

The computer system also includes user control, 27, such as may be a keyboard, pointing device, touch screen, remote interface including a wireless or internet connected device, including a mobile device. The user may be a physician, technician, nurse, or other user of the device.

The computer system also includes a display, 28, which may be wireless or remote.

Key embodiments include the ability of a user to see images generated by the both the camera, 16, and the ultrasound transducer together, as will be discussed more, below.

A key function of the computer system, 26, is to “stitch” the multiple images captured by the camera, 16, and ultrasound transducer, 17 to create one or more larger images without seams, or with minimized seams. Such electronic stitching of multiple images is well known in the art. For example, Adobe® Photoshop® software provides such capability. Other software includes open source “Hugin,” available from sourceforge.net and “Panotools,” available from the same source. The website: http://en.wikipedia.org/wiki/Comparison_of_photo_stitching_software provides a comparison of over 35 software packages that perform image stitching, all with links to sources.

Image stitching for ultrasound imagery is also known in the art. For example, the article, “Rapid image stitching and computer-aided detection for multipass automated breast ultrasound,” by Ruey-Feng Chang, Kuang-Che Chang-Chien, Etsuo Takada, Chiun-Sheng Huang, Yi-Hong Chou, Chen-Ming Kuo, Jeon-Hor Chen, published on ResearchGate, http://www.researchgate.net/publication/44653397_Rapid_image_stitching_and_computer-aided_detection_for_multipass_automated_breast_ultrasound, provides such information. This web site also provides a list of other technical publications describing image stitching of ultrasound images. Another publication on this topic is “3D Ultrasound Volume Stitching Using Phase Symmetry and Harris Corner Detection for Orthopaedic Applications,” by Rupin Dalvial Ilker Hacihaliloglu, Rafeef Abugharbieha at the Biomdedical Signal and Image Computing Laboratory, Department of Electrical and Computer Engineering, University Of British Columbia (UBC); available at: http://bisicl.ece.ubc.ca/focus/10_Rupin_SPIE.pdf.

Software to perform image stitching to generate 3D and VR images is also well known in the art. For example Autodesk®'s “123D Catch” software (http://www.123dapp.com/catch) converts a series of images taken around the perimeter of an object and returns a 3D file. 3D viewing software includes GLC Player, Sketchup, Rhino, and programs at http://3dfile.io. Some 3D file formats are listed above.

The computer system 26 typically provides for long-term storage and remote communication of images, as well as physician notes, not shown in the Figure.

Wireless communication from the sensors, 16 and 17, may be used, as well as with any interior electronics to control motion, 21, or provide sensor position information from inside the receptacle, 14, to the computer system 26. This has the advantage of fewer or now wires passing into or through the receptacle. Charging of batteries for the devices inside the receptacle may be accomplished via an electric field energy coupler (e.g., a transformer with the wall of the receptacle between the coils) with one coil proximal to the turntable 20 and coupling accomplished when the sensors or sensor assembly is in a “home” position to achieve effective electromagnetic coupling. This wireless communication, battery, charging circuit, and electromagnetic coupling are not shown in the Figure as they are well known in the art and thus there is no loss with this disclosure of scope for functional claim elements.

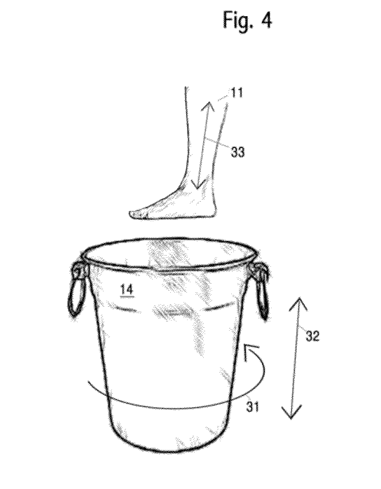

Turning now to FIG. 4, we see two preferred axes of scanning, 14 shows the receptacle, 11 shows a limb, here a foot and portion of a leg, 33 shows the axis of the limb. 31 shows rotation around the limb, 11, or around the limb axis, 33, in the transverse plane of the limb, 11, when the limb is placed in the receptacle, 14. 32 shows a line parallel to the axis of the limb, 33. The ideal scanning comprises effectively “looking in” from the interior surface of the receptacle, 14, towards entire surface of the limb, 11, except the portion of the surface of the limb, 11, that is facing the bottom of the receptacle. Often, less than this “100% scan” is available or used.

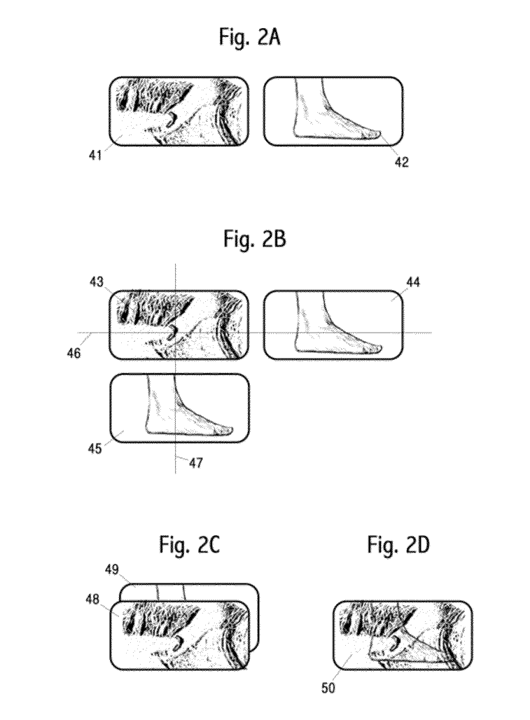

Turing now to FIGS. 2A through 2D, we see various embodiments of showing images from the ultrasound sensor and the camera displayed together. FIG. 2A shows an ultrasound image on the left, 41, and the camera image on the right, 42. Two separate displays may be used, or the images may be presented on a single physical display. Typically, multiple images from the sensors are “stitched,” as described above. This means that instead of a “flat” view, as shown in the Figure as 42, the images as seen on the display, such as 41, “wrap around” the limb. The physician may be able to control the choice of “flat” v. “wrap around” using the user control, 27.

FIG. 2B shows another embodiment. Here images are stacked vertically, rather than horizontally, shown as 43 and 45. Yet another embodiment shows both vertical and horizontal image comparison, 43, 44, and 45 together. Again, images may be presented on multiple display devices or on one.

A unique aspect and key embodiment is the ability for the physician to logically and diagnostically “align” a visual view of the surface of the limb with the deeper, subcutaneous ultrasound images. This may be used to help accurately locate pathologies visible in the ultrasound with either surface features, such as a bruise, or with known anatomical markers identifiable visually, such as the ankle bone (talus). In some treatments, the skin may be marked, such as with a tattoo, so that subsequent images may be accurately aligned with previously taken images, to show healing or treatment progress. In this way, markers may be used on the skin, yet effectively work as markers for ultrasound images. Previously, markers for ultrasound images were not available. Markers may include ink marks on the surface of the skin. Markers may include artificial elements below the skin such as screws or plates.

One embodiment that assists in the above alignment of visual and ultrasound features is the use of cross hairs, or similar display markers. These are shown as a horizontal cross hair, 46, and a vertical cross hair, 47. These are used to align a specific portion of the ultrasound image in 43 with visual anatomy in 44 and 45. Another embodiment uses both vertical and horizontal cross hairs on only two images, such are shown as 41 and 42. As the physician adjusts the desired location of the cross hairs, they move in parallel, or “track” across both or all displayed images.

In addition, in another embodiment, the physician or user may zoom, flip, pan vertically or horizontal, or rotate images via the user interface 27—again wherein the embodiment keeps both or images aligned. Other image enhancements, such as contrast, edge enhancement, false color and other display algorithms may be used, as those in the art know.

In FIG. 2C we see the ability to flip images alternately between the ultrasound 48 and the visible image, 49. Again, images are kept aligned, by the embodiment.

In FIG. 2D we see images from the two sensors overlaid, 50. Various overlay techniques maybe used, such as distinct colors, or different brightness ranges. The user may be able to adjust such display parameters, such as inverse video, of only one image at a time, in order to assist in differentiating features from one sensor with features from the other sensor. Again, images are kept aligned, by the embodiment.

Imagery in FIGS. 2A through 2D is purely schematic. These Figures do not accurately depict anatomical features or pathologies.

In some embodiments one or both the visual and ultrasound sensor are moved relative to the surface of the limb so as to maintain a constant, or a controlled, distance from the surface of the limb to the sensor. In one embodiment, the ultrasound sensor is in contact with the skin of the limb or on a sheath covering the limb. In another embodiment the optical camera comprises and autofocus element and that element serves both to control the focus of the camera and to provide distance information that is then used to position the ultrasound sensor as described in this paragraph.

In one embodiment the fluid or gel in the receptacle, 14, also serves to distribute light for the camera, 16. At least a portion of the inside surface of the receptacle, or a disposable bag inside the receptacle, is white or reflective to assist in the dispersal of such light.

In one embodiment a light source, such as one or more LED(s) or flash(es), is used as a light source for the camera, 16. In this embodiment, at least a portion of the inside surface of the receptacle, or a disposable bag inside the receptacle, is black or non-reflective such that most of the light used for visual imaging comes directly from the light source. This embodiment provides a high degree of uniformity and control over the light source for the visual images.

In one embodiment the limb is fully or partially immobilized during scanning. One method of immobilization is to use one or more inflatable balloons to surround the limb near the top of the receptacle, 14. Such an inflatable balloon may be a full or partial torus in shape. An alternative method of immobilization is the use of foam inserts, or other adjustable elements, manually inserted in place of the balloon(s).

In yet another embodiment, the limb is not immobilized. This has the advantage of convenience, speed, and minimal emotional trauma to the patient.

In yet another embodiment a shape adapted to the end of the limb is proximal to the bottom of the receptacle, 14, for example. A shape may be provided with one or more dimples or holes in which to place one or more fingers or fingertips. A shape be a ball or other shape suitable to be held in a hand. Another shape may be a full or partial inside base of a shoe, such as a heal-cup. Such shapes assist in holding the limb still during scanning, whether or not the limb is immobilized.

In one embodiment a turntable is used inside the receptacle that support the above-described shape. Thus, the receptacle may rotate, while both the limb and the support for the limb remain relatively fixed; as the base of the turntable rotates with the receptacle while the top of the turntable secures or steadies the end of the limb.

In one embodiment, 2D or 3D correlation is used by the computer system, 26, to align images taken at one time with images taken at a second time. Such image correlators are well known in the art. In another embodiment, changes between the early and later images are highlighted on the display, 26.

Some embodiments may use auto-measurement or auto-identification software to measure features or to provide feature identification. For example, the thickness of a bone may be measured with numerical data provided to the physician on the display using this embodiment. Or a break in a bone may be identified automatically and so indicated on the display. Blood flow, cavity size, and other measurements may be made automatically and the results displayed, aligned with the associated imagery.

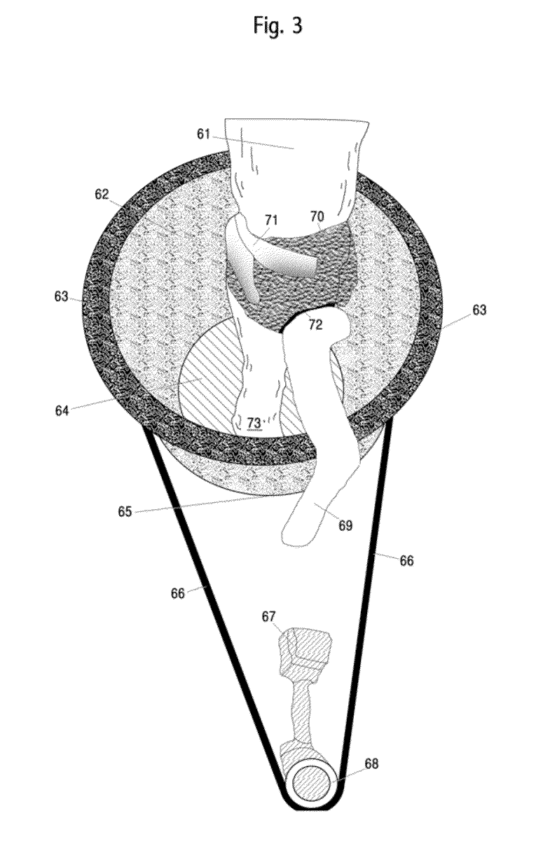

Turning now to FIG. 3, we see a schematic view of a portion of one embodiment in use. The receptacle or bucket is shown as 62. 63 is the upper rim of the bucket. 65 is the base of the bucket. The bucket, 62, is shown with a leg, 61, inserted. 73 identifies the foot on the leg, 61. In this Figure, the foot, 73, is resting on a turntable, 64. The foot, 73, may also be positioned or restrained by the use of a heal-cup, sandal, arch-contour, or similar anatomically matching shape, which is not shown in this Figure. In this embodiment, the receptacle 62 is rotated by means of a motor, 67, which drives a pulley wheel, 68, which in turn drives a belt 66.

In the embodiment shown in FIG. 3, the upper portion of the leg, 61, is restrained or secured by a knee cuff, 70, which is secured to the leg or knee with a closure, such as a hook and loop fastener, 71, and attached at point 72 to a clamp or guide 69. Alternative restraints include an inflatable balloon around all or a portion of the leg, inside the upper portion of the receptacle, 62.

In FIG. 3 no sensors, control electronics, power supplies, casings or enclosures, fluid, computer or display are shown. Any sheath used to cover the leg is not shown. Transparent fluid, or gel, such as water, in the receptacle, is not shown. A double bucket or receptacle within a receptacle may be used to isolate the sensors from the fluid; or isolate the limb from the sensors, or both. A double receptacle or double-wall receptacle is not shown in this Figure.

One application of this technology is to take measurements used in the creation of shoes, prosthetics, and other manufactured items in contact with and that work with the limb.

In the prior art, prosthetics and shoes were designed, sized and fitted base on the shape of the corresponding limb portion. That is, the shape as measured on the surface of the limb. Typically a “cast” is made of the limb to capture this surface shape. However, bone, muscle, nerves, tendons, scar-tissue, fat, cysts, joints, and other anatomical elements do not behave uniformly with respect to the performance of a shoe or prosthetic. Ideally, some portions of the shoe or prosthetic are firm, whereas others are soft or compliant. Ideally, some portions of the shoe or prosthetic should have fixed dimensions or shape, while other portions should be either compliant or adjustable. For example, bone is not (except in pre-adults) going to change size or shape at all, or at least not quickly. Whereas, muscle tissue will grow or shrink considerably due to exercise or atrophy. Nerves and tendons should not be underneath pressure points. Some portions of a limb expand and contract with fluid retention, or trauma, and thus may change size based on hydration, water-retention, monthly cycles, exercise, atrophy, or other known causes. Such areas of a shoe or prosthetic should be compliant or adjustable to adapt to this change in size. Other tissue types, such as fascia, can stretch with exercise. For example, distance runners find that their feet become longer during a race. In addition, anatomically, certain portions of a limb become the driving source or reference position for natural movement. Ideally, a shoe or prosthetic has its own positional or movement reference point based on this anatomical reference location.

With embodiments of this invention, the ability to create a 3D (or 4D, as will be discussed below) model of the limb, including its tissue types and shapes, permits a model basis and measurement basis for improved design, manufacturing or trimming (post-manufacturer fitting or adjustment) of the shoe or prosthetic. Thus, an embodiment uses the hardware and methods of this invention for the measurement of a fitted shoe, prosthetic, or accessory. Such accessories may include control devices, such as may be used in surgery, or to run a piece of equipment, including a vehicle or plane. For example, a custom stick could be created to exactly match a pilot's hand—not just the shape of the hand, but also the internal anatomy of the hand. A scan could be taken in a retail store or a doctor's office of a foot, and the measurements from that scan used to select, design, fabricate, adjust, or tune an orthotic, shoe, or accessory.

In the prior art, a “3D” scan might be only the surface, or shape of a limb. However, the 3D scans of embodiments of this invention also include the interior of the limb. A better description might be a “virtual reality” (or, VR) image, meaning a full set of 3D images, including depth and material, of the limb, viewable from any location, angle and distance.

Such scans, as described above, whether “3D” or “VR,” are nominally a fixed set of images of a static limb, in time.

However, some embodiments include additional sets of images based on movement of the limb. For example, a set of images of a let might include the knee bent at three different angles. The set might include images with the foot turned in pronation or supination. The set might include images with the leg standing on the toes of the foot, bearing weight, so as to show the positions of muscles, joints, and tendons in this active position. These additional images sets based on motion, movement, or activity may be thought of as a “fourth dimension” in additional to the normal 3D, three axes of information. Note that this fourth dimension of time or motion is also in addition to the depth and viewing information commonly associated with VR.

Yet another aspect of the scope of this fourth dimension of image information is based on time. In one aspect, time may be use to show progress of healing or progress of treatment. In another aspect, time may be used to show degeneration over time. In another aspect, time may be used on a short time scale to image movement, such as running, or squeezing and releasing a ball in a hand. Another aspect of time may be to show differences based on exercise or atrophy (such as before and after exercise); or water retention versus dehydration; or changes in blood pressure, include heartbeats.

On embodiment includes taking 3D, VR, or 4D measurements of a limb for the purpose of designing, fitting, or altering a prosthetic or shoe.

On embodiment includes taking 3D, VR, or 4D measurements of a limb as part of a process or method of designing, fitting, or altering a prosthetic or shoe.

One embodiment includes designing, manufacturing, fitting or altering a prosthetic or shoe responsive to the type of mating tissue as imaged, detected, or measured by the elements or methods described or claimed herein, including changes over time. For example, selecting responsively, the degree of: firmness, softness, compliance or adjustability of a portion of the prosthetic or shoe.

Suitable materials for construction of a prosthetic or shoe for the above embodiments include carbon-fiber shell; steel or stainless or aluminum structural elements; stretchy fabric or metal mesh; gel, foam; breathable fabrics; Hexcel® or other honeycomb or other composites; machined, cast or formed plastics, such as polycarbonate, adjustable fasteners such as hook & loop; hinges of all types; springs; electrically dynamic materials such as PZT or memory wire; or other materials.

Some embodiments use non-human animal limbs.

Embodiments of this invention explicitly include all combinations of all features and elements of all claims. Embodiments of this invention explicitly include devices and systems to implement any combination of all methods described in the claims, specification and drawings.

### Definitions

“Automatic restriction of the sensor scanning” refers to the speed of scanning (not too fast), and the spacing of adjacent scans (not too far apart). However, the operator of the device may select from the set of {starting location on the limb, scan direction, scan axis, resolution of scan, and limit of scan}.

“Changes in the images between the original scanning and rescanning are visible,” means that the images are displayed side-by-side; one above the other, superimposed, or compared electronically with a resulting difference image presented, or areas of differences between the images electronically determined and electronically highlighted.

“Driving a VR display” means, similar to “generate a display,” that the VR display may be local or remote, driven directly or indirectly, and that intermediate file formats, stored, queued, or transmitted in real time, may be used.

“Enclosure” may be the receptacle. Enclosure may be separate from the receptacle. The enclosure may be fully enclosed with the limb protruding, or may be open at one end.

“Generate a display” means to generate the necessary digital data, or electronic signals, or both, so as to drive a visual display, including either a local display or a remote display. A remote display may access the generated data via a web browser, for example. Other examples are generated image files of type jpeg, mpeg, flash, tiff, png, etc. Image data may be still image data, video data, flash or animation data, web data, or HTML5 data, for examples. Generate a display refers to either directly driving a physical display, such an LCD display, or providing real-time data for some other display device, or providing data that may be stored, queued, streamed, transmitted, or otherwise sent to a display indirectly in either space, or time, or both.

“Liquid” includes gel; the gel may be thicker than gel commonly used with ultrasound probes in the prior art.

“Sensors linked” means at least two sensors mechanically linked so that the relationship between the images generated by the sensors is known, for each imaged location on a limb; or electronically linked so that the relationship between the images generated by the sensors is known, for each imaged location on a limb

“Shape corresponding to at least a portion of the end of the limb refers to a shape designed or adapted to assist in either positioning, or restricting movement of the end of the limb, or both. Some such shapes are listed above, including a sandal or heal-cup for a foot, or holes for fingers or a ball for a hand.

“Substantially cylindrical enclosure” means an enclosure that is cylindrical over the portion of the enclosure that is used for the respective embodiment, and that is sufficiently cylindrical to work as described herein. For example, portions of the enclosure that are not used for imaging do not have to be cylindrical.

“Transparent” means transparent to the sensor or radiation. For example, a medium may be “transparent” to an ultrasound sensor, which means it passes the ultrasound waves or signals to and from the sensor sufficiently clear and with sufficient amplitude for the sensor to perform as designed or necessary the application of an embodiment of this invention. A medium that is transparent to IR light means that IR light may be used to form a suitable image through the medium using an IR sensitive sensor.

“Visibly aligned” means side-by-side, one above of the other, or superimposed views, such that the scale factors of the pair of images are the same.

Ideal, Ideally, Optimum and Preferred—Use of the words, “ideal,” “ideally,” “optimum,” “optimum,” “should” and “preferred,” when used in the context of describing this invention, refer specifically a best mode for one or more embodiments for one or more applications of this invention. Such best modes are non-limiting, and may not be the best mode for all embodiments, applications, or implementation technologies, as one trained in the art will appreciate.

May, Could, Option, Mode, Alternative and Feature—Use of the words, “may,” “could,” “option,” “optional,” “mode,” “alternative,” “typical,” “ideal,” and “feature,” when used in the context of describing this invention, refer specifically to various embodiments of this invention. Described benefits refer only to those embodiments that provide that benefit. All descriptions herein are non-limiting, as one trained in the art will appreciate.

I claim:

1. A method of medical imaging comprising the steps:
(a) placing a limb in a receptacle wherein the receptacle surrounds at least a portion of the limb;
(b) surrounding the portion of the limb with a transparent liquid;
(c) scanning, mechanically and automatically, the limb with a plurality of sensors comprising at least an optical camera and an ultrasound probe, wherein the scanning occurs in at least the transverse plane of the limb, or parallel to the axial axis of the limb, or both;
(c) obtaining both a series of visual images of the surface of the limb and a series of ultrasound images of the limb, the locations of the images of both series responsive to the scanning; wherein the obtaining of images by the plurality of sensors is linked such for each location on the limb, the visual image of that location is linked to a corresponding ultrasound image of the limb anatomically beneath the visual location;
(d) displaying at least one visual image and at least one corresponding ultrasound image wherein the two images are visibly aligned.

2. The method of claim 1 wherein the plurality of sensors are mechanically scanned both rotationally in the transverse plane of the limb and parallel to the axis of the limb.

3. The method of claim 1 wherein the transparent liquid is water.

4. The method of claim 1 wherein the transparent liquid is a gel suitable for use with the ultrasound probe.

5. The method of claim 1 wherein, during scanning in at least one axis, the distance of both the optical camera and the ultrasound prove are moved radially in the transverse plane of the limb, such that the sensors each maintain a respective constant distance between the surface of the limb and the sensor.

6. The method of claim 1 wherein the limb is not immobilized in the receptacle.

7. The method of claim 1 wherein the limb is immobilized in the receptacle.

8. The method of claim 1 wherein the limb is immobilized in the receptacle by an inflatable balloon.

9. The method of claim 1 wherein the end of the limb is positioned in the receptacle by an element comprising a shape corresponding to at least a portion of the end of the limb.

10. The method of claim 1 wherein the distance, in the transverse plane, between the optical camera and ultrasound probe, is mechanically fixed during scanning.

11. The method of claim 1 wherein the scanning in the transverse plane comprises at least 90° of rotation around the axis of the limb.

12. The method of claim 1 wherein at least one location of the plurality of sensors, relative to the limb, is controlled by an operator using a two-axis input device while a speed of scanning and a spacing of multiple, adjacent scans is automatically controlled.

13. The method of claim 1 wherein the limb is rescanned at a latter time, creating a latter series of visual images and a latter series of ultrasound images, and wherein at least a subset of the original visual and ultrasound images and a corresponding subset of the latter visual and ultrasound images are displayed such that changes in time between the original images and latter images are visible.

14. The method of claim 13 wherein the limb is visibly marked at one or more locations such that displayed original images and latter images are aligned to the same location on the limb, responsive to the visible marking.

15. The method of claim 13 wherein at least one image in the original series of images and at least one image in the latter series of images are electronically correlated using 2D or 3D mathematical correlation to find the most likely alignment between the original image and the latter images with respect to a location on the limb.

16. The method of claim 1 wherein the liquid is contained in a transparent enclosure aligned with the axis of the limb and wherein the optical camera is located outside the enclosure and the ultrasound probe is located inside the transparent enclosure.

17. The method of claim 1 wherein the liquid is contained in a transparent, enclosure aligned with the axis of the limb and wherein the optical camera is located outside the enclosure and the ultrasound probe is located outside the enclosure and is in contact with the outside surface of the enclosure.

18. The method of claim 1 wherein the scanning comprises at least one magnet external to the receptacle and at least one magnet internal to the receptacle and the external magnet is driven and the internal magnet tracks the external magnet.

19. The method of claim 1 comprising the additional step:
(e) transmitting digital image data from at least one of the sensors wirelessly.

20. The method of claim 1 comprising the additional step:
(f) charging a battery internal to the receptacle via electromagnetic coupling through the receptacle.

21. The method of claim 1 comprising the additional step:
(g) driving a virtual reality (VR) display comprising medical imaging information the limb comprising: (i) a surface location on the surface of the limb; (ii) medical image data of the surface of the limb at the surface location; (iii) medical image data under the skin of the limb at the surface location; (iv) ability of an operator to control a viewing source location for simultaneous viewing of at least (ii) and (iii).

22. The method of claim 1 comprising the additional step:
(h) automated measurement of at least one anatomical feature responsive to both a visual image and a corresponding ultrasound image.

23. The method of claim 1 comprising the additional step:
(j) automated detection of at least one anatomical pathology responsive to both a visual image and a corresponding ultrasound image.

24. The method of claim 1 comprising the additional step:
(k) enclosing the limb in a sanitary, optically transparent, sheath prior to placing the limb in the receptacle.

25. The method of claim 1 wherein:
the receptacle is a double-wall receptacle comprising an interior and exterior wall with a gap in between the two walls; wherein the plurality of sensors are located between the interior and exterior walls; and wherein the transparent liquid and the limb are in the interior of the interior wall.

26. The method of claim 1 comprising the additional steps of:
(m) autofocusing the optical camera such that an optical distance from the focal plane of the optical camera to the location on the surface of the limb being imaged by the optical camera is obtained;
(n) moving at least one sensor radially in the transverse plane of the limb, responsive to the optical distance, such that a constant distance between the sensor and the surface of the limb being imaged by that sensor, is obtained.

27. The method of claim 1 wherein the limb is immobilized at its distal end and not immobilized at its proximal end.

28. A device for implementing the method of claim 1.

29. A device for medical imaging a limb comprising:
a receptacle adapted to contain at least a portion of a limb;
a transparent liquid in the receptacle;
a first sensor comprising an optical camera;
a second sensor comprising an ultrasound probe;
a mechanical scanning mechanism adapted to scan the first and second sensors at least one of: (i) rotationally around the transverse plane of the limb, and (ii) parallel to the axial axis of the limb;
wherein the optical camera generates a series of visual images of the limb; and wherein the ultrasound probe generates a series of ultrasound images of the limb; wherein the series of images correspond to different locations on the limb, as imaged by the two sensors, responsive to the scanning;
a computer system;
a computer program in non-transitory memory in the computer system, adapted to generate a display of a subset of the visual images and a subset of the ultrasound images visibly aligned such that each visual imaged location on the surface of limb is visibly aligned with the ultrasound image anatomically beneath the visually imaged location.

30. The device of claim 29 further comprising:
a computer program in non-transitory memory in the computer system, adapted to stitch of a first subset of the visual images and to stitch a second subset of the ultrasound images such that a continuous stitched image comprising the first subset and a continuous stitched image comprising the second subset are available for viewing, visibly aligned to each other with respect to the anatomy of the limb.

31. The device of claim 29 further comprising:
a turntable adapted to rotate the receptacle.

32. The device of claim 29 further comprising:
a magnetic coupling adapted to drive the sensors magnetically with the magnetic field penetrating the wall of the receptacle.

33. The device of claim 29 further comprising:
a wireless transmitter adapted to transmit at least a portion of digital image data from at least one sensor.

34. The of claim 29 further comprising:
an electromagnetic coupling adapted to charge a battery interior to the receptacle, wherein the electromagnetic field penetrates the wall of the receptacle.
